(19) United States
(12) Patent Application Publication
(10) Pub. No.: US 2012/0109110 A1
(43) Pub. Date: May 3, 2012

(54) SUCTION CATHETER WITH RETRACTABLE BLADE

(76) Inventor: JOSEPH T. SOWDER, Pelham, AL (US)

(21) Appl. No.: 12/915,220

(22) Filed: Oct. 29, 2010

(51) Int. Cl. A61M 27/00; A61B 17/3211

(52) U.S. Cl. 604/543; 606/167

(57) ABSTRACT

The present invention in several embodiments is a device comprising a retractable scalpel blade within the barrel of a plastic suction catheter, which can be attached to a wall suction canister found in most exam rooms. The device would facilitate the procedure of abscess drainage by allowing one to incise the abscess with the scalpel blade when it is extended out from the end of the barrel of the suction catheter, then retract the blade into the barrel tip after the appropriate incision is made. Then the blunt tip of the catheter can be used to suction out the abscess contents, as well as break up the loculations within the abscess cavity, and also subsequently suction out saline if it is used for irrigation of the cavity.

### FIELD OF THE INVENTION

The invention is related to surgical devices. The present invention is particularly applicable to suction catheters and scalpel blades and combinations thereof.

### BACKGROUND OF THE INVENTION

The has been a need for a device that can simplify and speed the process of draining abscesses, either in an emergency department or in a physician's office.

Accordingly, it has been considered desirable to develop a new and improved suction catheter which would overcome the foregoing difficulties and others while providing better and more advantageous overall results.

### SUMMARY OF THE INVENTION

The present invention in several embodiments is a device comprising a retractable scalpel blade within the barrel of a plastic suction catheter, which can be attached to a wall suction canister found in most exam rooms. The device would facilitate the procedure of abscess drainage by allowing one to incise the abscess with the scalpel blade when it is extended out from the end of the barrel of the suction catheter, then retract the blade into the barrel tip after the appropriate incision is made. Then the blunt tip of the catheter can be used to suction out the abscess contents, as well as break up the loculations within the abscess cavity, and also subsequently suction out saline if it is used for irrigation of the cavity.

The present invention could be made so that the scalpel blade is initially covered, the cover removed, and the blade then retracted after use, or it could be made so that the blade can be advanced and retracted at will from the end of the catheter. The end of the catheter may or may not contain fenestrations. The catheter body is preferably constructed of a rigid material such as polycarbonate. The blade could be any suitable blade, however a number 11 scalpel blade is currently envisioned.

Some of the advantages of the present invention over the prior art include a single device that is used for incising as well as drainage aspects of medical procedures; a self-contained retractable blade to decrease the risk of inadvertent scalpel injuries; a hard plastic catheter and tip that can be used to de-loculate the abscess; a disposable one piece device that obviates the need for an incision and drainage kit (I&D kit) that contains several instruments; the ability to remove the abscess contents with suction; the ability to virtually eliminate the malodorous condition caused by the opening of the abscess (by virtue of the suction), as well as simplifying the clean up process. The decreased time and effort needed for this procedure using such a device would allow for increased time, and improved patient satisfaction. The present invention would also be ideal for the drainage of peritonsilar abscesses and Bartholin gland abscesses, in addition to subcutaneous abscesses.

It is envisioned that the present invention could be manufactured to be contained in sterilized prepackaged packets. It is also envisioned that the present invention could be manufactured to be a disposable device.

### OBJECTS OF THE INVENTION

It is therefore one of the primary objects of the present invention to provide an integrated gutter, drain and corner cap system.

Another object of the present invention is to provide integrated gutter, drain and corner cap system that is resistant to clogging.

In one embodiment the invention is a suction catheter for removing a substance from a body vessel, comprising: an elongated catheter tube having a proximal end and a distal end, a means for connecting to a suction means located at said proximal end, a blade attached to said distal end. In another embodiment said blade is a #11 scalpel blade. In still another embodiment said blade is a retractable blade. In yet another embodiment said blade is capable of being retracted into an interior space of said suction catheter. In still yet another embodiment said suction catheter is substantially shaped as a Yankauer suction catheter. In yet still another embodiment said blade is capable of being retracted into an interior space of said suction catheter, wherein said blade is constructed of steel, and wherein said elongated catheter tube is constructed of polycarbonate. In yet another embodiment said blade is capable of being retracted at will by a user into an interior space of said suction catheter, wherein said blade is constructed of steel, and wherein said elongated catheter tube is constructed of polycarbonate.

In one embodiment the invention is a suction catheter for removing a substance from a body vessel, comprising: an elongated Yankauer catheter tube having a proximal end and a distal end, a means for connecting to a suction means located at said proximal end, a retractable scalpel blade attached to said distal end. In another embodiment said retractable scalpel blade is capable of being retracted into an interior space of said suction catheter. In yet another embodiment said retractable scalpel blade is capable of being retracted at will by a user into an interior space of said suction catheter.

It is to be understood that both the foregoing general description and the following detailed description are merely exemplary of the invention, and are intended to provide an overview or framework for understanding the nature and character of the invention as it is claimed. The accompanying drawings are included to provide a further understanding of the invention, and are incorporated in and constitute a part of this specification. The drawings illustrate various embodiments of the invention; and together with the description serve to explain the principles and operation of the invention.

### BRIEF DESCRIPTION OF THE DRAWINGS

FIG. 1 is a general perspective view drawing of a preferred embodiment of the present invention with the blade in a retracted position.

FIG. 2 is a general perspective view drawing of a preferred embodiment of the present invention with the blade in an extended position.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

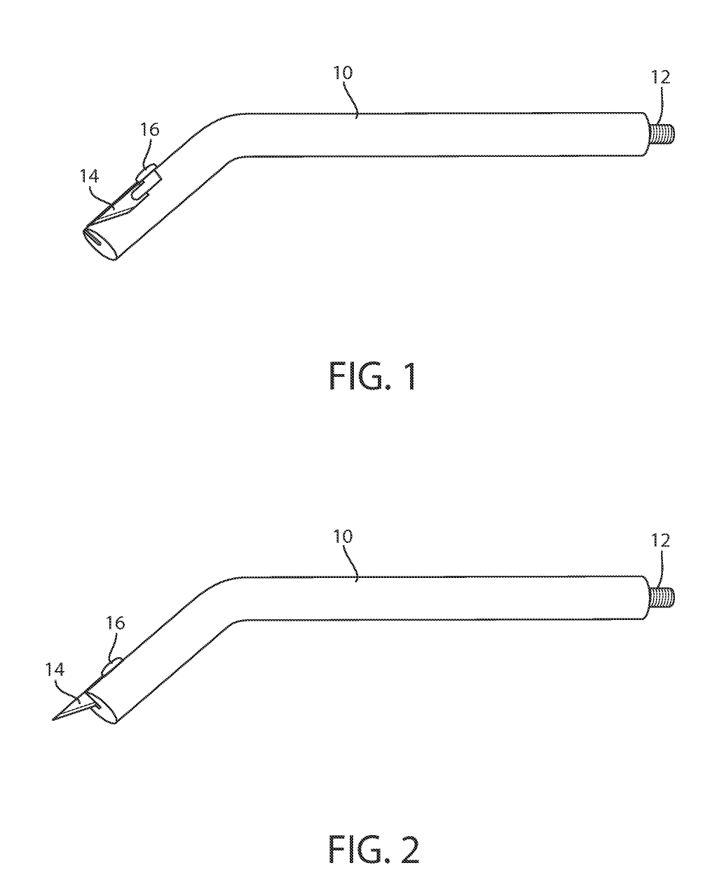

Reference is now made, more particularly, to FIG. 1 which is a general perspective view drawing of a preferred embodiment of the present invention with the blade in a retracted position.

The present invention is a device comprising a suction catheter 10, attachment means to a suction canister 12, scalpel blade 14, and retraction mechanism 16.

Reference is now made, more particularly, to FIG. 2 which is a general perspective view drawing of a preferred embodiment of the present invention with the blade in an extended position. The present invention is a device comprising a suction catheter 10, attachment means to a suction canister 12, scalpel blade 14, and retraction mechanism 16.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

1. A suction catheter for removing a substance from a body vessel, comprising:
an elongated catheter tube having a proximal end and a distal end,
a means for connecting to a suction means located at said proximal end,
a blade attached to said distal end.

2. The suction catheter of claim 1, wherein said blade is a #11 scalpel blade.

3. The suction catheter of claim 1, wherein said blade is a retractable blade.

4. The suction catheter of claim 1, wherein said blade is capable of being retracted into an interior space of said suction catheter.

5. The suction catheter of claim 1, wherein said suction catheter is substantially shaped as a Yankauer suction catheter.

6. The suction catheter of claim 1, wherein said blade is capable of being retracted into an interior space of said suction catheter, wherein said blade is constructed of steel, and wherein said elongated catheter tube is constructed of polycarbonate.

7. The suction catheter of claim 1, wherein said blade is capable of being retracted at will by a user into an interior space of said suction catheter, wherein said blade is constructed of steel, and wherein said elongated catheter tube is constructed of polycarbonate.

8. A suction catheter for removing a substance from a body vessel, comprising:
an elongated Yankauer catheter tube having a proximal end and a distal end,
a means for connecting to a suction means located at said proximal end,
a retractable scalpel blade attached to said distal end.

9. The suction catheter of claim 8, wherein said retractable scalpel blade is capable of being retracted into an interior space of said suction catheter.

10. The suction catheter of claim 8, wherein said retractable scalpel blade is capable of being retracted at will by a user into an interior space of said suction catheter.
